(19) United States
(12) Patent Application Publication
(10) Pub. No.: US 2021/0121435 A1
(43) Pub. Date: Apr. 29, 2021

(54) METHODS FOR TREATING SEIZURES IN EPILEPSY WITH A COMPOSITION COMPRISING CANNABINOIDS

(71) Applicants: MEDRELEAF CORP., Toronto (CA); THE GOVERNING COUNCIL OF THE UNIVERSITY OF TORONTO, Toronto (CA)

(72) Inventors: Shannon O'HEARN, Toronto (CA); Alexia BLAKE, Toronto (CA); Istok NAHTIGAL, Toronto (CA); Angelo FEFEKOS, Toronto (CA); McIntyre Willets BURNHAM, Toronto (CA); Junhan Liu, Toronto (CA)

(21) Appl. No.: 16/746,576

(22) Filed: Jan. 17, 2020

Related U.S. Application Data
(60) Provisional application No. 62/927,526, filed on Oct. 29, 2019.

(51) Int. Cl. A61K 31/352; A61K 31/05; A61K 36/185; A61K 9/00; A61K 45/06; A61P 25/08

(52) U.S. Cl. CPC A61K 31/352; A61K 31/05; A61P 25/08; A61K 9/0053; A61K 45/06; A61K 36/185

(57) ABSTRACT

Methods for treating seizures in a subject having epilepsy are provided. The method may comprise administering a pharmaceutical composition comprising CBD and THC to the subject. In some embodiments, the ratio of CBD to THC in the pharmaceutical composition is between about 14:1 and about 17:1. Related uses of the pharmaceutical composition to treat seizures in a subject having epilepsy are also provided.

### RELATED APPLICATION

The present application claims priority to U.S. Provisional Patent Application No. 62/927,526, filed Oct. 29, 2019, the entire contents of which are incorporated herein by reference.

### FIELD OF THE DISCLOSURE

The present disclosure relates to treatment of epilepsy. More particularly, the present disclosure relates to treatment of seizures in patients with epilepsy with a composition comprising cannabidiol (CBD) and Δ⁹-tetrahydrocannabidiol (THC).

### BACKGROUND

Epilepsy is a chronic disorder of the central nervous system that affects 0.5 to 1% of the population. While anti-epileptic drugs (AEDs) are the most commonly used therapy for epilepsy, about 30% of people with epilepsy do not achieve seizure control (1, 2). A person is typically considered to have drug-resistant epilepsy if they are not seizure-free after trying two appropriate AEDs.

Drug-resistant epilepsy impacts all aspects of life, including psychological aspects (depression, anxiety), social aspects (stigma, isolation), and economic factors (restricted job access, economic hardship) (3,4). Long-term health risks include neuropsychiatric conditions (5), cognitive impairment including memory problems (6,7), reproductive and endocrine disorders (8), and an increased risk of premature death (9).

Over 60% of epilepsies are classified as idiopathic, where extensive investigation has been unable to identify the cause (10). Evidence suggests that genetic factors may contribute to the observed differences in response to the various anti-epileptic treatments (11).

Cannabis refers to plants of the Cannabis genus. The Cannabis genus is generally understood to comprise one species, Cannabis sativa L., although some botanical authorities also recognize Cannabis indica and Cannabis ruderalis. Cannabis plants produce a variety of chemical compounds including cannabinoids which may have biological activity in humans and other organisms.

Cannabis and its cannabinoid-rich extracts appear to have anti-seizure activity. The two main cannabinoids typically extracted from the Cannabis plant are cannabidiol (CBD) and Δ⁹-tetrahydrocannabinol (THC). Both CBD and THC appear to have anti-convulsive activity; however, THC may also have intoxicating effects that limit its tolerability.

CBD and THC have been the subject of preclinical animal studies. Studies related to THC have produced ambiguous results—a review of 31 preclinical studies found that THC was anticonvulsant in 19 studies, had no effect in 9 studies and was proconvulsive in 3 studies (17). Preclinical studies of CBD have generally shown anticonvulsant effects—in 21 different studies, 17 reported anticonvulsant effects and none reported proconvulsive effects (17).

Recent clinical trials have examined the ability of CBD to reduce seizures in children and young adults with epilepsy (12-15). The best controlled study of the anticonvulsive effects of CBD in humans is a recent study involving participants with Dravet Syndrome and drug-resistant seizures. CBD significantly reduced the frequency of occurrence of convulsive seizures, and about 5% of patients became seizure free (12). A follow-up study in participants with Dravet Syndrome (DS), Lennox-Gastaut Syndrome (LGS) and intractable epilepsy was performed. At 12 weeks, patients with DS had a median reduction of 49.8% and 4 participants were free of seizures in the last four weeks of treatment. Patients with LGS had a median reduction of 36.8%, and 1 participant was free of seizures in the last four weeks of treatment (13).

Cunha et al (24) is a study in which 15 patients with temporal-lobe epilepsy and at least one generalized convulsion per week were randomized to receive either CBD or placebo. Of the 7 participants who received CBD, 4 were seizure free during treatment, while only 1 out of 8 in the control group was seizure free (24,25). In study conducted by Hess et al. (15), participants with tuberous sclerosis complex (TSC) and treatment-resistant epilepsy were administered CBD. After 3 months of treatment the median percent change in total weekly seizure frequency was −48.8%.

Few controlled clinical trials have examined the ability of THC to reduce seizures in humans. Moreover, most preclinical studies have studied CBD and THC separately and their interaction with regards to anti-convulsive activity is not well understood. As a result, the safety and efficacy of specific Cannabis extracts, and specific ratios of CBD to THC, is difficult to predict.

### SUMMARY

In one aspect, there is provided a method for treating seizures in a subject having epilepsy, the method comprising administering a pharmaceutical composition comprising cannabidiol (CBD) and Δ⁹-tetrahydrocannabinol (THC) to the subject, wherein the ratio of CBD to THC in the pharmaceutical composition is between about 14:1 and about 17:1.

In some embodiments, the ratio of CBD to THC in the pharmaceutical composition is between about 15:1 and about 16:1.

In some embodiments, the pharmaceutical composition comprises a Cannabis extract.

In some embodiments, the Cannabis extract is from the Avidekel™ cultivar.

In some embodiments, the Cannabis extract is a whole plant extract.

In some embodiments, the pharmaceutical composition further comprises cannabiochromene (CBC).

In some embodiments, the pharmaceutical composition further comprises at least one terpene.

In some embodiments, the subject is an adult.

In some embodiments, the seizures are drug-resistant seizures.

In some embodiments, the subject has at least one comorbidity of epilepsy and the pharmaceutical composition treats the at least one comorbidity.

In some embodiments, the at least one comorbidity comprises at least one of depression, anxiety, and Autism Spectrum Disorder.

In some embodiments, the pharmaceutical composition is administered concomitantly with at least one other anti-seizure medication.

In some embodiments, the subject is not concurrently taking another anti-seizure medication and the pharmaceutical composition is administered to the subject as a monotherapy.

In some embodiments, the pharmaceutical composition is administered to the subject at a CBD dosage of between about 100 mg/day and about 300 mg/day.

In some embodiments, the pharmaceutical composition is administered to the subject at a THC dosage of between about 5 mg/day and about 20 mg/day.

In some embodiments, the pharmaceutical composition is administered orally.

In some embodiments, the pharmaceutical composition is administered parenterally.

In another aspect, there is provided a kit comprising a pharmaceutical composition comprising CBD and THC in a container and prescribing information that includes instructions for taking the pharmaceutical composition to treat seizures, wherein the ratio of CBD to THC in the pharmaceutical composition is between about 14:1 and about 17:1.

In some embodiments, the prescribing information includes instructions for use of the pharmaceutical composition with at least one other medication.

In some embodiments, the at least one other medication comprises an anti-seizure medication.

Other aspects and features of the present disclosure will become apparent, to those ordinarily skilled in the art, upon review of the following description of specific embodiments of the disclosure.

### BRIEF DESCRIPTION OF THE DRAWINGS

Some aspects of the disclosure will now be described in greater detail with reference to the accompanying drawings. In the drawings:

FIG. 1A is a line graph showing percentage suppression of generalized seizures in amygdala-kindled rats treated with various doses of CBD or a combination of CBD and THC (approximately 15:1 CBD:THC);

FIG. 1B is a line graph showing percentage suppression of focal seizures in amygdala-kindled rats treated with various doses of CBD or a combination of CBD and THC (approximately 15:1 CBD:THC);

FIG. 2A is a line graph showing percentage suppression of generalized seizures in amygdala-kindled rats treated with various doses of THC alone;

FIG. 2B is a line graph showing percentage suppression of focal seizures in amygdala-kindled rats treated with various doses of THC alone; and

FIG. 3 is a bar graph showing percentage suppression of focal seizures in amygdala-kindled rats treated with 160 mg/kg CBD, 10 mg/kg THC, or a combination of 160 mg/kg CBD and 10.33 mg/kg THC (approximately 15:1 CBD:THC).

### DETAILED DESCRIPTION

Generally, the present disclosure provides a method for treating seizures in a subject having epilepsy. The method may comprise administering a pharmaceutical composition comprising CBD and THC to the subject. A related use of the pharmaceutical composition to treat seizures in a subject having epilepsy is also provided.

The pharmaceutical composition used in the method disclosed herein may comprise cannabidiol (CBD) and Δ⁹-tetrahydrocannabinol (THC). Embodiments herein also contemplate any possible enantiomers, isomers, and mixtures thereof of CBD and THC.

The pharmaceutical composition may have a high ratio of CBD to THC. In some embodiments, the ratio of CBD to THC is between about 20:1 and about 10:1, or between about 18:1 and about 12:1, between about 17:1 and about 14:1, or between about 16:1 and about 15:1. In embodiments, the ratio of CBD:THC is about 17:1, about 16:1, or about 15:1.

In some embodiments, the CBD concentration in the pharmaceutical composition is between about 10% and about 20%, or between about 12% and about 18%, or between about 11% and about 15%, or about 12% (by weight). In some embodiments, the THC concentration is between about 0.1% and about 1.2%, or between about 0.2% and about 1%, or between about 0.3% and about 0.9%, or about 0.8% (by weight).

In some embodiments, the pharmaceutical composition may comprise one or more additional cannabinoids. In some embodiments, the composition further comprises (±)-cannabichromene (CBC). In some embodiments, the ratio of CBD to CBC is between about 20:1 and about 10:1, or between about 18:1 and about 12:1, or between about 17:1 and about 14:1. In some embodiments, the composition comprises between about 0.1% and about 1.2% CBC (by weight).

In some embodiments, the pharmaceutical composition may further comprise minor amounts of one or more other cannabinoids including but not limited to: cannabigerol (CBG), cannabinol (CBN) and/or cannabidivarol (CBDV). In some embodiments, such minor cannabinoids may be present at about 0.2% or less of the composition. In other embodiments, the composition may comprise any other cannabinoid.

In some embodiments, the pharmaceutical composition may further comprise one or more terpenes. In some embodiments, the composition may comprise at least one of beta-myrcene, alpha-pinene, limonene, beta-pinene, and guaiol. In other embodiments, the composition may comprise any other terpene. Terpenes may act synergistically with cannabinoids to enhance their effects in a theory known as the “entourage effect”.

In some embodiments, the pharmaceutical composition may further comprise one or more flavonoids or any other chemical compound found in Cannabis plants.

In some embodiments, the pharmaceutical composition comprises an extract from a Cannabis plant (hereafter referred to as a “Cannabis extract”). As used herein, a “Cannabis plant” refers to a plant of the Cannabis genus. In some embodiments, the Cannabis plant is Cannabis sativa L. In some embodiments, the Cannabis plant is Cannabis sativa L. subspecies indica. In other embodiments, the Cannabis plant is any other species or subspecies of the Cannabis genus.

In some embodiments, the Cannabis plant is of the Avidekel™ cultivar available from MedReleaf™ or Tikun Olam™. The Avidekel cultivar is described in more detail in U.S. patent application Ser. No. 15/633,166, published as US20170290286A1, incorporated herein by reference. Other embodiments of Cannabis extracts useful herein may be from the cultivar Trutiva™, available from MedReleaf™, or from the cultivar Rafael™, available from MedReleaf™ or Tikun Olam™. In other embodiments, the Cannabis plant may be any suitable variety or cultivar.

Within Cannabis plants, CBD mainly occurs in the form of cannabidiolic acid (CBDA) and THC mainly occurs in the form of Δ⁹-tetrahydrocannabinolic acid (THCA). In some embodiments, the Cannabis plant has a high ratio of CBDA to THCA.

The Cannabis extract may be produced from Cannabis plant material from one or more Cannabis plants. In some embodiments, the Cannabis plant material comprises at least one of floral material (including flowers, buds, trichome heads and/or trichomes), leaves, seeds, stems, and combinations thereof, from one or more Cannabis plants. In some embodiments, the Cannabis plant material comprises flowerings heads. In some embodiments, the Cannabis plant material is dried. The Cannabis plant material may be dried using any suitable drying method, including but not limited to, air-drying or drying in a drying tumbler. In some embodiments, the Cannabis plant material is milled. The Cannabis plant material may be milled using any suitable milling method including dry- or wet-milling methods.

In some embodiments, the Cannabis extract is a whole plant extract. As used herein, a “whole plant extract” refers to an extract containing both the extractable cannabinoid and terpene content of the Cannabis plant material (and potentially other chemical compounds), the composition of which may depend on the type of extraction process used. Whole plant extracts may be produced from any suitable Cannabis plant material and are not limited to extraction of an entire Cannabis plant.

In some embodiments, the whole plant extract may be produced using supercritical fluid extraction. The supercritical fluid may comprise supercritical carbon dioxide (CO2), for example. Briefly, CO2 may be pressurized to form a liquid and the liquid may be mixed with dried and milled cannabis plant material to extract the desired compounds. The first stage of the extraction recovers the terpene fraction and the second stage of the extraction recovers the cannabinoid fraction. The carbon dioxide may dissipate off the collected fractions at the end of the extraction process and therefore little to no carbon dioxide may be present in the final extract.

In some embodiments, the cannabinoid fraction is decarboxylated by the application of heat. As used herein, “decarboxylated” or “decarboxylation” refers to a treating the cannabinoid fraction in a manner to convert at least a portion of THCA to THC and CBDA to CBD. In some embodiments, approximately all of the THCA and CBDA in the original plant material are converted to THC and CBD, such that there is little to no THCA and CBDA present in the cannabinoid fraction. In other embodiments, minor amounts of THCA and/or CBDA may remain in the cannabinoid fraction, for example, less than 15% or less than 10% of the total THC (THC plus THCA) or total CBD (CBD plus CBDA).

The terpene and cannabinoid fractions may then be combined to produce the whole plant extract. Alternatively, the fractions may be combined first and then subjected to decarboxylation as described above. The terpene content may be determined by gas chromatography (GC) and the cannabinoid content may be determined by high performance liquid chromatography (HPLC). In some embodiments, the terpene and cannabinoid fractions are combined at the same ratios at which they were recovered in the extraction process. In other embodiments, the terpene and cannabinoid fractions may be combined at any other suitable ratio based on desired cannabinoid and/or terpene concentrations in the final extract.

In other embodiments, the Cannabis extract may be produced by solvent extraction (e.g. ethanol extraction), hydrocarbon extraction (e.g. butane or propane extraction), cold water extraction, etc. In some embodiments, the Cannabis extract may be subjected to one or more refinement processes, including but not limited to winterization and/or distillation.

In other embodiments, the Cannabis extract may be produced by a solvent-free extraction method. As one example, solvent-free extraction may involve separating the trichome heads of the Cannabis plant from the remainder of the plant matter (e.g. by sieving dry cannabis or by agitating Cannabis in ice water). In some embodiments, the separated trichomes are exposed to heat and/or pressure to form a resin.

In other embodiments, the pharmaceutical composition may comprise dried Cannabis plant material having the desired cannabinoid ratios described above.

In other embodiments, the pharmaceutical composition may comprise a combination of synthetic and/or purified CBD and synthetic and/or purified THC, combined in the ratios described above. In some embodiments, the synthetic/purified CBD or THC is more than 98% pure, as determined by high performance liquid chromatography (HPLC) or any other suitable method. In some embodiments, the pharmaceutical composition may further comprise synthetic and/or purified CBC and/or any other suitable synthetic/purified cannabinoid, terpene, or any other suitable chemical compound.

In other embodiments, the pharmaceutical composition may comprise a combination of a Cannabis extract and synthetic/purified CBD and/or THC. For example, the Cannabis extract may be supplemented with synthetic/purified CBD and/or THC as needed to arrive at the desired ratios as described above.

In some embodiments, the pharmaceutical composition may be formulated for oral administration. The pharmaceutical composition may be in the form of a pill, a capsule, a tablet, a liquid (including an edible oil), a powder or granule, a spray, a dissolvable strip, or any other suitable form for oral administration. In some embodiments, the pharmaceutical composition may further comprise at least one pharmaceutically or nutritionally acceptable excipient. Non-limiting examples of suitable excipients include fillers, binders, carriers, diluents, stabilizers, lubricants, glidants, coloring agents, flavoring agents, coatings, humectants, disintegrants, preservatives, sorbents, sweeteners and any other pharmaceutically or nutritionally acceptable excipient. In embodiments in which the composition is in the form of a capsule, the composition may further comprise a suitable encapsulation material, including but not limited to polyvinyl alcohol (PVA), polyvinylpurrolidone (PVP), alginates, gelation, etc.

In an embodiment the composition additionally comprises high oleic sunflower oil, or another oil having similar chemical properties. Sunflower oil may reduce allergen potential, ensure oxidative stability, increase batch to batch consistency, and improve bioavailability of CBD and THC.

In other embodiments, the pharmaceutical composition may be formulated for parenteral administration. The pharmaceutical composition may be formulated for injection intravenously, intramuscularly, subcutaneously, intradermally, or intraperitoneally. The composition may comprise one or more pharmaceutically acceptable sterile aqueous or nonaqueous solutions, suspensions, emulsions, or sterile powders which may be reconstituted into sterile injectable solutions or dispersions prior to use. Such solutions or powders may contain antioxidants, buffers, stabilizing agents, preservatives, and/or solutes which render the formulation isotonic with the blood of the intended recipient, etc.

In other embodiments, the pharmaceutical composition may be formulated for topical or transdermal administration including a powder, spray, ointment, paste, cream, lotion, gel, solution, patch, drop etc. In other embodiments, the pharmaceutical composition may be for administration by inhalation or insufflation including formulations suitable for delivery by an inhaler, nebulizer, insufflator, etc. In other embodiments, the pharmaceutical composition may be formulated for any other suitable type of administration and embodiments are not limited by the specific dosage forms described herein.

The pharmaceutical composition can be an immediate-, fast-, slow-, sustained-, or delayed-release or any other suitable type of composition.

Provided herein is a method of treating seizures in a subject with the pharmaceutical composition described above. Also provided herein is a use of the pharmaceutical composition in treating seizures in a subject. In some embodiments, the subject is a mammalian subject. In some embodiments, the subject is a human. In embodiments, the subject may be an adult (i.e. 18 years or older).

In some embodiments, the subject has epilepsy. As used herein, “epilepsy” refers to a disorder of brain function characterized by the periodic occurrence of seizures. Epilepsy refers to a clinical phenomenon rather than a single disease entity, since there are many forms and causes of epilepsy. A subject may be diagnosed as having epilepsy when they have two or more unprovoked seizures.

As used herein, “seizure” includes convulsive seizures and non-convulsive seizures. The term “convulsive” seizure includes tonic, tonic-clonic, atonic, drop attacks, and focal motor seizures. The term “non-convulsive” seizure includes absence seizures. The seizures may be generalized or focal seizures. The term “generalized” seizure includes seizures that affect both hemispheres of the brain simultaneously e.g. tonic clonic, myoclonic, or absence seizures. The term “focal” seizure includes seizures that affect one hemisphere, lobe, or focal area e.g. complex partial seizure.

As used herein, “treat” or “treatment” refers to obtaining a desired pharmacologic and/or physiologic effect. The effect can be prophylactic in terms of completely or partially preventing a health condition or symptom thereof and/or can be therapeutic in terms of partial or complete cure for the health condition and/or adverse effect attributable to the health condition. As used herein, “treat” or “treatment” of seizures in a subject refers to reducing the frequency and/or severity of seizures in the subject.

In some embodiments, the subject has drug-resistant seizures, including drug-resistant convulsive seizures and drug-resistant non-convulsive seizures. In preferred embodiments, the methods disclosed herein are used to treat adults that have drug-resistant convulsive seizures.

“Drug-resistant seizures” may alternately be referred to as uncontrolled, intractable, or refractory seizures. In this disclosure, a subject is considered to have “drug-resistant” seizures when they have failed to become (and stay) seizure free with adequate trials of at least two anti-seizure medications. The terms “anti-seizure” medication, “anti-convulsive” medication or “anti-convulsant”, and “anti-epileptic” drug (AED) or medication are all used herein to refer to any medication that is used to treat seizures. For a diagnosis of drug-resistant seizures, the two or more medications: have been chosen appropriately for the person's seizure type; have been tolerated by the person; and have been tried alone or in combination with other anti-seizure medications (International League Against Epilepsy definition). In embodiments, a person with “drug-resistant seizures” experiences at least four seizures in a four-week period prior to the start of treatment, while taking prescribed anti-epileptic drugs.

In some embodiments, the pharmaceutical composition may also be used to treat at least one comorbidity of epilepsy. The term “comorbidity” in this context refers to the presence or one or more additional health conditions or disorders that co-occur with the primary condition (epilepsy). In some embodiments, the comorbidity comprises depression, anxiety, or Autism Spectrum Disorder. In other embodiments, comorbidity may comprise any other health condition or disorder co-occurring with epilepsy.

The methods disclosed herein are directed to the administration of a therapeutically effective amount of CBD and THC. In some embodiments, the therapeutically effective amount may be administered as a single dose per day. In other embodiments, the therapeutically effective amount may be administered in two or more sub-doses at appropriate intervals throughout the day, or as microdoses throughout the day. While it is preferred that the CBD and THC be administered together as one dose, embodiments herein contemplate separate administration of the two compounds.

The dosage of CBD used in the methods described herein may be between about 100 mg/day and about 500 mg/day, between about 100 mg/day and about 300 mg/day, or between about 200 mg/day and 300 mg/day. In embodiments the dosage of CBD is about 100 mg/day, about 200 mg/day or about 300 mg/day. Preferably the dosage is about 300 mg/day.

The dosage of THC used in the methods described herein may be less than about 20 mg/day and less than about 10 mg/dose, to avoid psychotropic effects from THC when a dose is taken. The dosage of THC used in the methods described herein may be between about 5 mg/day and about 20 mg/day, between about 10 mg/day and about 20 mg/day, or between about 15 mg/day and 20 mg/day. In embodiments the dosage of THC is about 5 mg/day, about 10 mg/day or about 20 mg/day with no one dose greater than 10 mg THC.

In embodiments the dosage of CBD used in the methods described herein is between about 1.4 mg/kg/day and about 7.1 mg/kg/day, between about 1.4 mg/kg/day and about 4.3 mg/kg/day, or between about 2.9 mg/kg/day and 4.3 mg/kg/day. In embodiments the dosage of CBD is about 1.4 mg/kg/day, about 2.9 mg/kg/day or about 4.3 mg/kg/day. Preferably the dosage is about 4.3 mg/kg/day.

In embodiments the dosage of THC used in the methods described herein may be less than about 0.29 mg/kg/day and less than about 10 mg/dose, to avoid psychotropic effects from THC when a dose is taken. The dosage of THC used in the methods described herein may be between about 0.07 mg/kg/day and about 0.29 mg/kg/day, between about 0.14 mg/kg/day and about 0.29 mg/kg/day, or between about 0.21 mg/kg/day and 0.29 mg/kg/day. In embodiments the dosage of THC is about 0.14 mg/kg/day, about 0.21 mg/kg/day or about 0.29 mg/kg/day (with no one dose greater than 10 mg THC).

In some embodiments, the pharmaceutical composition may be administered orally. In other embodiments, the pharmaceutical composition may be administered parenterally such as intravenously, intramuscularly, subcutaneously, intradermally, or intraperitoneally. In other embodiments, the pharmaceutical composition may be administered topically, transdermally, by inhalation or insufflation, or by any other suitable route of administration.

In some embodiments, the pharmaceutical composition may be administered as a monotherapy. As used herein, “monotherapy” refers to use of a single medication in the treatment of a specific symptom or health condition. In some embodiments, the pharmaceutical composition may be administered as a monotherapy for seizures. When the pharmaceutical composition is used as a monotherapy for seizures, the subject may not be concurrently taking another anti-seizure medication, although the subject may be taking other medication(s) for other symptoms including pain, depression, anxiety, etc.

In other embodiments, the pharmaceutical composition may be administered as a combination therapy or an adjunctive therapy. As used herein, “combination therapy” refers to use of two or more medications to treat a specific symptom or health condition. As used herein, “adjuvant therapy” refers to use of a secondary medication in addition to a primary medication to treat a specific symptom or health condition. For example, the pharmaceutical composition may be administered as an adjuvant therapy in addition to one or more anti-seizure medications.

The pharmaceutical composition may be administered concomitantly with one or more other medications. In some embodiments, the pharmaceutical composition may be formulated for administration separately, sequentially, or simultaneously with the other medication(s). In some embodiments, where the pharmaceutical composition and other medication are administered simultaneously, the pharmaceutical composition may be provided in a single dosage form with the other medication.

In some embodiments, the pharmaceutical composition may be administered concomitantly with at least one anti-seizure/anti-epileptic medication. Non-limiting examples of anti-seizure/anti-epileptic medications include: phenytoin, stiripentol, clobazam, topiramate, valproic acid, lamotrigine, levetiracetam, clonazepam, clobazamphenytoin, phenobarbital, carbamazepine, oxcarbazepine, gabapentin, pregabalin, lacosamide, vigabatrin, zonisamide, and rufinamide.

In some embodiments, the pharmaceutical composition may be administered concomitantly with at least one other medication for a comorbidity of epilepsy such as, for example, a medication for depression, anxiety, or Autism Spectrum Disorder. In other embodiments, the pharmaceutical composition may be administered concomitantly with any other suitable medication e.g. a pain medication.

Also provided herein is a kit comprising a pharmaceutical composition in a container and prescribing information that includes instructions for taking the pharmaceutical composition to treat seizures. The pharmaceutical composition may be any embodiment of the pharmaceutical composition described above.

In some embodiments, the prescribing information can include instructions for use of the pharmaceutical composition with at least one other medication. The instruction may include, for example, instructions to take the composition and other medication(s) at the same or at different times, with or without food, etc.

In some embodiments, the other medication comprises an anti-seizure medication. The anti-seizure medication may be any of the anti-seizure/anti-epilepsy medications described above. In other embodiments, the other medication may comprise at least one other medication for a comorbidity of epilepsy such as, for example, a medication for depression, anxiety, or Autism Spectrum Disorder. In other embodiments, the other medication may be any other suitable medication.

Without any limitation to the foregoing, the present compositions and methods are further described by way of the following examples.

### EXAMPLES

### Preclinical Study

Preclinical studies were performed to study the effects of CBD or THC alone, or CBD in combination with THC, on seizures. Amygdala kindled rats were used as a model for generalized and focal seizures in these studies.

Kindling Procedure: An insulated bipolar electrode was implanted in the right basolateral amygdala of male Sprague Dawley rats (50-55 days old). Two weeks following implantation, the rats began daily kindling. The stimulation used for kindling was a 1 second train of biphasic 60 Hz square wave pulses at an intensity of 400 micro-amps, once per day. Both the focal afterdischarge (AD) and convulsive behavior were monitored for a minimum of 30 seconds after stimulation or until an observed seizure ended. Rats were stimulated once daily until ten stage 5 Racine scale seizures have been generated, after which they are considered fully kindled (typically 3-4 weeks).

Seizure Scoring: The focal seizures were defined by observing the afterdischarge (AD) recorded by electroencephalogram (EEG) via the implanted electrode. The focal AD can last as long as 1-2 minutes post stimulation. The focal AD is not necessarily accompanied by a secondary generalized motor seizure, especially early in the kindling process. Early behavioural signs may include the cessation of behaviour or blinking. After sufficient kindling has been induced, generalized motor events of seizure generalization began to occur. These events were rated using the Racine scale. The stages are as follows: Stage 1—facial clonus; Stage 2—head nodding; Stage 3—forelimb clonus; Stage 4—forelimb clonus and rearing on hindlimbs; Stage 5—loss of postural control (rearing and following over).

Seizure Threshold Testing: Two days after the 10^{th} stage 5 seizure was recorded, the animal's seizure threshold was tested using ascending series technique. The process was started at an intensity of 20 micro-amps, which was increased by 20 micro-amps every two minutes until an AD was produced. The seizure threshold is defined as the current needed to produce the AD.

Stability Testing: After seizure thresholds were determined, the animals underwent stability testing to confirm the reliability of the threshold. The animals were stimulated at 25% above the determined threshold 3 times per week for 2 weeks. If seizures were consistently evoked at this level of stimulation, the rat was considered stable. Spontaneous seizures do not typically occur.

Drug Testing: Once the animals were successfully stability tested, the drug testing commenced. Stimulation testing occurred 2 hours after the intraperitoneal (IP) injection of CBD, THC, or a combination of CBD and THC. The combination dosage required 2 injections. The vehicle control for all studies was a combination of ethanol (5%), Cremophore EL (5%) and phosphate buffered saline (overall ratio of 1:1:18).

The effects of: a) CBD alone (0, 20, 40, 80, 160, 320 mg/kg; 10 subjects per dose); b) THC alone (0, 5, 10, 20, 40, 80 mg/kg; 10 subjects per dose); or c) CBD+THC (15:1) (CBD dose of 0, 20, 40, 80, 160, 320 mg/kg; 10 subjects per dose), were tested. The drug treatments were kept at a constant volume (3 mL CBD and 2 mL THC). The CBD-treated rats were stimulation tested at 2 hours post-injection and the THC-treated rats were tested at 1 hour post-injection. A generalized seizure was considered to be suppressed if there was an absence of stage 3 or higher seizure, assessed using the Racine scale. A focal seizure was considered to be suppressed if there was an absence of EEG seizure activity lasting more than 4 seconds.

Before each round of drug testing stimulation, the rats were assessed for motor toxicity using the Loscher ataxia scale. This scale is used to determine motor impairment. The scale rankings are as follows: Stage 1—slight ataxia in hind legs, no decrease in abdominal muscle tone; Stage 2—more pronounced ataxia, slight decrease in abdominal muscle tone, flat body position; Stage 3—increased ataxia, continued decrease in abdominal muscle tone, more pronounced flat body posture, splayed hind legs, and crawling during forward motion; Stage 4—marked ataxia, loss of balance during forward motion, loss of abdominal muscle tone, flat body posture, splayed hind legs, and dragging during forward motion; Stage 5—very marked ataxia, frequent loss of balance during forward motion, loss of abdominal muscle tone.

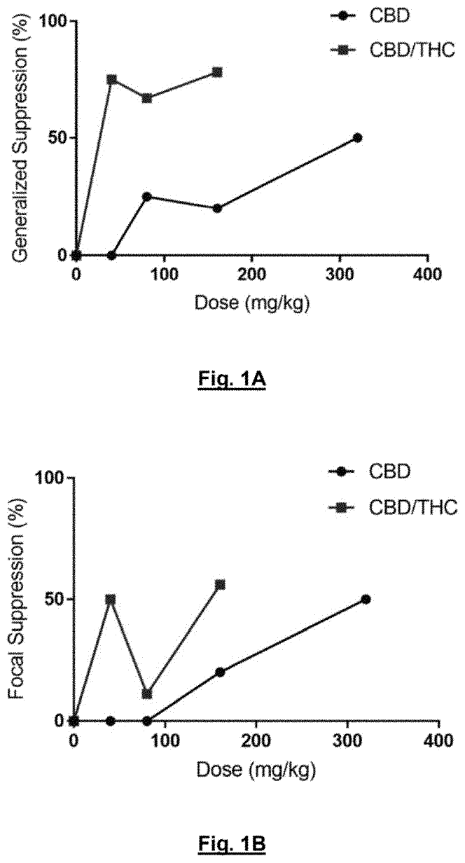

As shown in FIGS. 1A and 1B, CBD alone was found to suppress some generalized and focal seizures. CBD had no observed toxic effects up to 320 mg/kg.

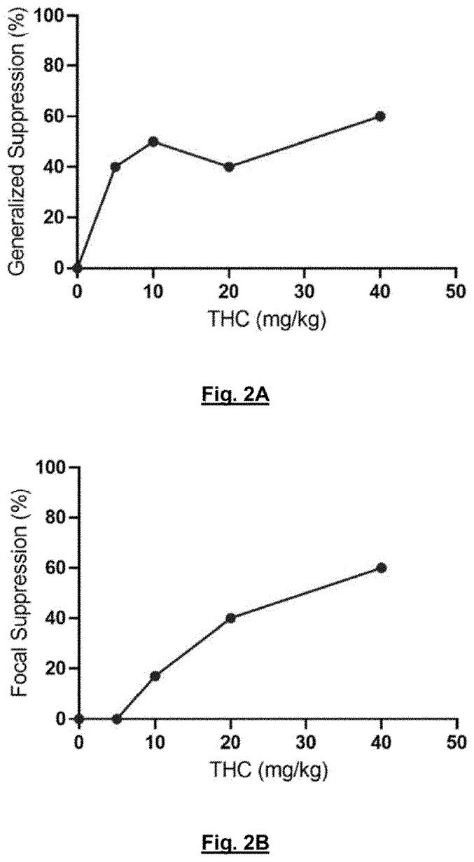

As shown in FIGS. 2A and 2B, THC alone was found to suppress some generalized and focal seizures. However, psychotoxicity was observed at doses above 10 mg/kg.

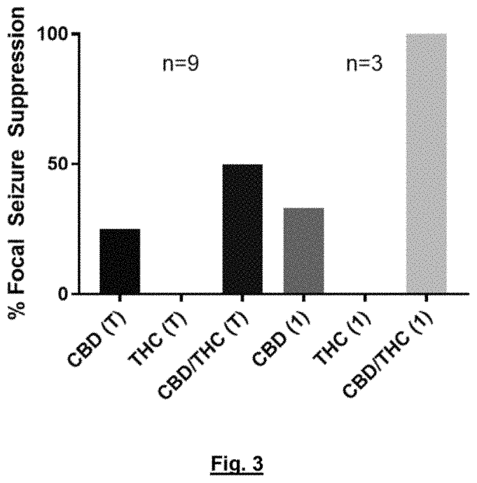

Referring again to FIGS. 1A and 1B, the combination of CBD and a small amount of THC (at approximately a 15:1 ratio) shifted the CBD curve to the left for both generalized and focal seizures. FIG. 3 summarizes the percentage of focal seizure suppression at 160 mg/kg CBD, 10 mg/kg THC, and 160 mg/kg CBD with 10.33 mg/kg THC (˜15:1 CBD:THC). As shown in FIG. 3, there is a synergistic effect of the combination of CBD and THC on the suppression of focal seizures. The combination of CBD and THC, therefore, may allow for more complete suppression of seizures as compared to the equivalent dose of CBD alone, and at doses of THC that are not psychotoxic.

### Clinical Study

A double-blind, placebo-controlled, parallel-group study of cannabidiol plus tetrahydrocannabinol (CBD+THC) given as adjunctive therapy in patients with refractory seizures was planned as follows.

The study population consists of adults (18 years of age or older) with drug-resistant convulsive seizures. To be included, the participants meet the following criteria: a diagnosis of epilepsy according to the ILAE (International League Against Epilepsy) classification; at least 4 convulsive seizures per month at the start of the study, despite treatment with at least two different anti-epileptic drugs (given concurrently or sequentially) for at least one year; at least 4 convulsive seizures during the 4-week baseline period of the study with no 21-day seizure free periods; and stable dose(s) of the same anti-epileptic drug for one month prior to screening. The patient group will include patients with Dravet syndrome, patients with Lennox-Gastaut Syndrome, and patients with any form of epilepsy involving frequent convulsive seizures.

Convulsive seizures include tonic, tonic-clonic, atonic, drop attacks and focal motor seizures. The study focuses on convulsive seizures for clarity in recording, and on patients with frequent seizures, so that anti-seizures effects will be relatively evident. It is anticipated that a reduction in frequency observed with respect to convulsive seizures would also be observed with non-convulsive seizures.

Excluded from the study are:
patients where there is evidence of clinically significant non-epileptic disease (cardiac, respiratory, gastrointestinal, hepatic, hematologic, or renal disease, etc.) that could affect the patient's safety or trial conduct;
patients with progressive central nervous system (CNS) disease, including degenerative CNS diseases and progressive tumors;
patients who have experienced psychogenic seizures in the year before the start date;
patients with a history of drug misuse/abuse (other than cannabinoids) within a defined relevant time period;
patients with multiple drug allergies (dermatological, hematological, or organ toxicity) or more than one severe drug reaction(s),
pregnant or breastfeeding patents,
patients with known or suspected hypersensitivity to cannabinoids, or any of the excipients of the investigational medicinal product;
patients with a history of major depression, suicidal ideation or attempted suicide, schizophrenia or any other psychotic disorder; and
patients with a family history of schizophrenia.

A total of 80 participants will be included in the study: 40 in the treatment group and 40 in the control group. The study will be a Phase III, double-blind, randomized, placebo-controlled add-on trial, followed by an open phase where all participants receive the study drug.

The participants are maintained on their existing antiepileptic medications. Common antiepileptic medications include phenytoin, stiripentol, clobazam, topiramate, valproic acid, lamotrigine, levetiracetam, clonazepam, clobazamphenytoin, phenobarbital, carbamazepine, oxcarbazepine, gabapentin, pregabalin, lacosamide, vigabatrin, zonisamide, and rufinamide.

The study drug is a capsule for oral administration comprising a Cannabis extract. Each capsule comprises CBD and THC in a ratio of approximately 16:1. As CBD and THC are hydrophobic, the extract is diluted with high oleic sunflower oil, to a concentration of about 250 mg CBD/ml of oil. Each capsule of study drug contains about 50 mg CBD (55 mg Total CBD, which is CBD and CBDA), and about 3 mg THC (3.5 mg THC and THCA).

At a dose of 300 mg/day of CBD, participants would receive about 18 mg of THC per day, 9 mg in the morning and 9 mg in the evening. According to the literature, there may be psychotropic effects at a dose of 10 mg of THC (41).

The placebo consists of high oleic sunflower calorically matched to the CBD dose and packaged in identical capsules.

Clinical trials of CBD have demonstrated that CBD is safe and well tolerated across a wide dosage range. No significant adverse effects have been reported for doses up to 1500 mg/day (p.o.) (14,23). Devinsky et al. (12) tested CBD in children and young adults with Dravet syndrome. The dose utilized was 20 mg/kg/day, which in a 70 kg adult male would be equivalent to a CBD dose of 1,400 mg/day. Other clinical studies have employed CBD doses of 200-300 mg/day in epilepsy (24,32). For other indications (anxiety, multiple sclerosis, schizophrenia) doses have ranged from 150-1500 mg CBD per day (23). The dose of CBD for the study will be 300 mg/day, administered in two separate doses.

The Cannabis extract used in the study will be a whole plant extract from the Avidekel™ cultivar. Neither THC nor CBD is present in raw Cannabis material. Rather, they are found in their acidic forms, THCA and CBDA. Decarboxylation is required to convert the acidic forms to THC and CBD, which is accomplished by heat treatment of the extract.

Whole plant extract is prepared from dried and milled trim material using supercritical fluid extraction, with carbon dioxide as the supercritical fluid. The first stage of the extraction recovers the terpene fraction and the second stage of the extraction recovers the cannabinoid fraction. The terpene content is determined by gas chromatography (GC) and the cannabinoid content is determined by high performance liquid chromatography (HPLC). The cannabinoid fraction is decarboxylated by the application of heat.

Over 200 terpenes have been identified in the Cannabis plant, and they may interact synergistically with cannabinoids in an extract, to enhance their effects. A pre-clinical study (63) compared the anti-inflammatory and anti-nociceptive properties of purified CBD to whole-plant CBD obtained from the Avidekel strain (15-17% CBD to <1% THC). The two treatments were administered to mice either intraperitoneally or orally. A bell-shaped dose-response was observed following administration of purified CBD, meaning that therapeutic benefits were observed within a narrow dose range. In comparison, therapeutic benefits increased with doses of whole-plant CBD extract. The authors suggested that other extract components, such as terpenes, may interact synergistically with CBD to offer superior efficacy that is not dose-limited.

All participants will maintain stable dose(s) of their existing medications throughout the duration of the study. The duration of the study will be 16 weeks for the treatment group and 18 weeks for the control group, divided into the following periods/phases.

Baseline Period (4 weeks): the initial baseline phase for both groups, where no study drug or placebo are administered. Participants with prior use of Cannabis (cannabis-users) will undergo an additional pre-trial washout period for 4 weeks prior to the Baseline Period.

Titration Period (2 weeks): the control group will receive placebo, and the treatment group will receive 100 mg/day CBD for the first week, and 200 mg/day CBD for the second week. The capsules are administered twice per day. If any adverse side effects are seen in a participant in the treatment group, the dose will not be increased and participant may either titrate down to the lowest dose (100 mg of CBD per day) or stop treatment.

Treatment Period (4 weeks): the control group will continue to receive placebo, and the treatment group will receive 300 mg CBD/day, or the highest tolerated dose. At a dose of 300 mg/day of CBD, participants will receive 18 mg of THC per day, 9 mg in the morning and 9 mg in the evening.

Maintenance Period (4 to 6 weeks): Participants in the control group will receive the study drug for the same duration as the treatment group (2 week titration, 4 week treatment). Participants in the treatment group will receive the study drug for an additional 4 weeks.

Washout Period (2 weeks): participants receive 200 mg/day CBD for the first week and 100 mg/day CBD for the second week.

The half-life of CBD, administered orally, may be about 1 to 2 days. A steady state is may be reached about 10 days after regular dosing begins (i.e., 4 to 5 times the half-life). As such, with a 4 week treatment period all patients may achieve a steady state of CBD blood concentration.

The primary objective of the study will be to evaluate the effects of oral CBD:THC (16:1) on convulsive seizure frequency in adult patients with refractory epilepsy maintained on standard antiepileptic medications. The secondary objectives of the study are to assess the safety profile of CBD:THC (16:1), to assess CBD and THC blood levels following oral dosing, to assess any impact on blood levels of antiepileptic drug (AED) and liver enzymes, to determine any impact on cognition, emotion and/or quality of life. More particularly, the following will be measured:
% change in frequency of convulsive seizures in the Treatment Period, as compared to the Baseline Period (in treatment and control groups);
% change in frequency of convulsive seizures in the Maintenance Period, as compared to the Baseline Period and Treatment Period (in treatment and control groups);
incidence and severity of reported adverse events in treatment group, as compared to control group;
change in serum concentration of CBD and THC during the Treatment Period/Maintenance Period relative to the Baseline Period for the treatment and control groups (blood samples will be collected after the Baseline Period, after the Treatment Period and after the Maintenance Period);
change in concentration of Anti-Epileptic Drugs (AEDs) and liver enzymes in serum from Baseline Period to Treatment Period to Maintenance Period in treatment and control groups; and
change in score from Baseline Period to Treatment Period to Maintenance Period in quality of life, quality of sleep, depressive symptoms, anxiety, global impression of change, overall severity of epilepsy, psychological and functional impairment, in the treatment group, as compared with control.

Seizure frequency in all Periods will be assessed by a patient seizure diary. Participants will record their frequency of seizures and side effects; the journal will be collected and monitored weekly. In the event of an adverse event (AE) or serious adverse event (SAE), participants and their caregivers will be required to report the events within 24 hours of occurrence to the research assistant/coordinator.

Study questionnaires/CDEs will be administered at the end of the Baseline Period. Common Data Elements (CDEs will be collected online three times throughout the study: after the Baseline Period, after the Treatment Period, and after the Maintenance Period (Control group only).

The results from this study will provide families, physicians, and the epilepsy community with clinical data regarding dosing, safety, and efficacy of CBD+THC in adults with refractory seizures.

While the methods, uses, and compositions have been described in conjunction with the disclosed embodiments which are set forth in detail, it should be understood that this is by illustration only and the disclosure is not intended to be limited to these embodiments. On the contrary, this disclosure is intended to cover alternatives, modifications, and equivalents which will become apparent to those skilled in the art in view of this disclosure.

### REFERENCES

The following references are hereby incorporated by reference in their entirety:
1. Kwan P, Brodie M J. Early identification of refractory epilepsy. N Engl J Med. 2000 Feb. 3; 342(5):314-9.
2. Dodson W. Efficacy. In: Engel J, Pedley T, editors. Epilepsy: A Comprehensive Textbook. New York: Lippincott-Raven Publishers; 1997. p. 1155-65.
3. Kobau R, Gilliam F, Thurman D J. Prevalence of self-reported epilepsy or seizure disorder and its associations with self-reported depression and anxiety: results from the 2004 HealthStyles Survey. Epilepsia. 2006 November; 47(11):1915-21.
4. Laxer K D, Trinka E, Hirsch L J, Cendes F, Langfitt J, Delanty N, et al. The consequences of refractory epilepsy and its treatment. Epilepsy Behav E B. 2014 August; 37:59-70.
5. Gaitatzis A, Carroll K, Majeed A, W Sander J. The epidemiology of the comorbidity of epilepsy in the general population. Epilepsia. 2004 December; 45(12):1613-22.
6. Pharmacologic Treatment. Epilepsia. 2003; 44(s6):33-4.
7. McAuley J W, Elliott J O, Patankar S, Hart S, Long L, Moore J L, et al. Comparing patients' and practitioners' views on epilepsy concerns: a call to address memory concerns. Epilepsy Behav E B. 2010 December; 19(4):580-3.
8. Herzog A G, Fowler K M. Sexual hormones and epilepsy: threat and opportunities. Curr Opin Neurol. 2005 April; 18(2): 167-72.
9. Duncan J S, Sander J W, Sisodiya S M, Walker M C. Adult epilepsy. Lancet Lond Engl. 2006 Apr. 1; 367(9516):1087-100.
10. WHO|Epilepsy [Internet]. WHO. [cited 2017 Sep. 14]. Available from: http://www.who.int/mediacentre/factsheets/fs999/en/11.
11. Sisodiya S M. Genetics of drug resistance in epilepsy. Curr Neurol Neurosci Rep. 2005 July; 5(4):307-11.
12. Devinsky O, Cross J H, Laux L, Marsh E, Miller I, Nabbout R, et al. Trial of Cannabidiol for Drug-Resistant Seizures in the Dravet Syndrome. N Engl J Med. 2017 25; 376(21):2011-20.
13. Devinsky O, Marsh E, Friedman D, Thiele E, Laux L, Sullivan J, et al. Cannabidiol in patients with treatment-resistant epilepsy: an open-label interventional trial. Lancet Neurol. 2016 March; 15(3):270-8.
14. Devinsky O, Cilio M R, Cross H, Fernandez-Ruiz J, French J, Hill C, et al. Cannabidiol: pharmacology and potential therapeutic role in epilepsy and other neuropsychiatric disorders. Epilepsia. 2014 June; 55(6):791-802.
15. Hess E J, Moody K A, Geffrey A L, Pollack S F, Skirvin L A, Bruno P L, et al. Cannabidiol as a new treatment for drug-resistant epilepsy in tuberous sclerosis complex. Epilepsia. 2016 October; 57(10):1617-24.
16. Gaston T E, Friedman D. Pharmacology of cannabinoids in the treatment of epilepsy. Epilepsy Behav E B. 2017 May; 70(Pt B):313-8.
17. Hill A J, Hill T D., Whalley B J. The development of cannabinoid based therapies for epilepsy. In: Murillo-Rodriguez E, editor. Endocannabinoids: molecular, pharmacological, behavioral and clinical features bentham science publishers, Oak Park, I L. Bentham Science Publishers; 2013. p. 164-204.
18. Boggan W O, Steele R A, Freedman D X. 69-Tetrahydrocannabinol effect on audiogenic seizure susceptibility. Psyehopharmacologia Berl. 1973; 29:101-6.
19. Corcoran M E, McCaughran J A, Wada J A. Acute antiepileptic effects of 9-tetrahydrocannabinol in rats with kindled seizures. Exp Neurol. 1973 August; 40(2):471-83.
20. Klein B D, Jacobson C A, Metcalf C S, Smith M D, Wilcox K S, Hampson A J, et al. Evaluation of Cannabidiol in Animal Seizure Models by the Epilepsy Therapy Screening Program (ETSP). Neurochem Res. 2017 July; 42(7):1939-48.
21. Jones N A, Hill A J, Smith I, Bevan S A, Williams C M, Whalley B J, et al. Cannabidiol displays antiepileptiform and antiseizure properties in vitro and in vivo. J Pharmacol Exp Ther. 2010 February; 332(2):569-77.
22. Jones N A, Glyn S E, Akiyama S, Hill T D M, Hill A J, Weston S E, et al. Cannabidiol exerts anti-convulsant effects in animal models of temporal lobe and partial seizures. Seizure. 2012 June; 21(5):344-52.
23. Bergamaschi M M, Queiroz R H C, Zuardi A W, Crippa J A S. Safety and side effects of cannabidiol, a Cannabis sativa constituent. Curr Drug Saf. 2011 Sep. 1; 6(4):237-49.
24. Cunha J M, Carlini E A, Pereira A E, Ramos O L, Pimentel C, Gagliardi R, et al. Chronic administration of cannabidiol to healthy volunteers and epileptic patients. Pharmacology. 1980; 21(3):175-85.
25. Gloss D, Vickrey B. Cannabinoids for epilepsy. Cochrane Database Syst Rev. 2014 Mar. 5; (3):CD009270.
26. Cannabinoid Therapy for Pediatric Epilepsy—Full Text View—ClinicalTrials.gov [Internet]. [cited 2017 Jul. 28]. Available from: https://clinicaltrials.gov/ct2/show/NCT02983695
27. Cannabidiol in Children With Refractory Epileptic Encephalopathy—Full Text View—ClinicalTrials.gov [Internet]. [cited 2017 Jul. 28]. Available from: https://clinicaltrials.gov/ct2/show/NCT03024827
28. Mathern G W, Beninsig L, Nehlig A. Fewer specialists support using medical marijuana and CBD in treating epilepsy patients compared with other medical professionals and patients: result of Epilepsia's survey. Epilepsia. 2015 January; 56(1):1-6.
29. Szoeke C E I, Newton M, Wood J M, Goldstein D, Berkovic S F, OBrien T J, et al. Update on pharmacogenetics in epilepsy: a brief review. Lancet Neurol. 2006 February; 5(2):189-96.
30. Balestrini S, Sisodiya S M. Pharmacogenomics in epilepsy. Neurosci Lett. 2017 Jan. 10;
31. Treat L, Chapman K E, Colborn K L, Knupp K G. Duration of use of oral cannabis extract in a cohort of pediatric epilepsy patients. Epilepsia. 2017 January; 58(1):123-7.
32. Ames F R, Cridland S. Anticonvulsant effect of cannabidiol. South Mr Med J Suid-Afr Tydskr Vir Geneeskd. 1986 Jan. 4; 69(1):14.
33. Vaney C, Heinzel-Gutenbrunner M, Jobin P, Tschopp F, Gattlen B, Hagen U, et al. Efficacy, safety and tolerability of an orally administered cannabis extract in the treatment of spasticity in patients with multiple sclerosis: a randomized, double-blind, placebo-controlled, crossover study. Mult Scler Houndmills Basingstoke Engl. 2004 August; 10(4):417-24.
34. Wade D T, Robson P, House H, Makela P, Aram J. A preliminary controlled study to determine whether whole-plant cannabis extracts can improve intractable neurogenic symptoms. Clin Rehabil. 2003 February; 17(1):21-9.
35. Wade D T, Makela P, Robson P, House H, Bateman C. Do cannabis-based medicinal extracts have general or specific effects on symptoms in multiple sclerosis? A double-blind, randomized, placebo-controlled study on 160 patients. Mult Scler Houndmills Basingstoke Engl. 2004 August; 10(4):434-41.
36. Bergamaschi M M, Queiroz R H C, Chagas M H N, de Oliveira D C G, De Martinis B S, Kapczinski F, et al. Cannabidiol reduces the anxiety induced by simulated public speaking in treatment-naïve social phobia patients. Neuropsychopharmacol Off Publ Am Coll Neuropsychopharmacol. 2011 May; 36(6):1219-26.
37. Lev-Ran S, Le Foll B, McKenzie K, Rehm J. Cannabis use and mental health-related quality of life among individuals with anxiety disorders. J Anxiety Disord. 2012 December; 26(8):799-810.
38. Weeks C. Canadian doctors given guidelines on prescribing marijuana—The Globe and Mail. The Globe and Mail [Internet]. 2014 Sep. 29 [cited 2017 Aug. 24]; Available from: https://www.theglobeandmail.com/life/health-and-fitness/health/doctors-to-get-guidelines-on-prescribing-marijuana/article20836275/
39. Gregorio J. Physicians, Medical Marijuana, and the Law. Virtual Mentor. 2014 Sep. 1; 16(9):732-8.
40. Devinsky O, Burnham W M. CBD. 2017.
41. Grotenhermen F. Pharmacokinetics and pharmacodynamics of cannabinoids. Clin Pharmacokinet. 2003; 42(4):327-60.
42. Health Canada. Information for Health Care Professionals: Cannabis (marihuana, marijuana) and the cannabinoids [Internet]. Health Canada; 2013 [cited 2017 Dec. 19]. Available from: https://www.canada.ca/en/health-canada/services/drugs-health-products/medical-use-marijuana/information-medical-practitioners/information-health-care-professionals-cannabis-marihuana-marijuana-cannabinoids.html#chp32
43. Geffrey A L, Pollack S F, Bruno P L, Thiele E A. Drug-drug interaction between clobazam and cannabidiol in children with refractory epilepsy. Epilepsia. 2015 August; 56(8):1246-51.
44. Gaston T, Bebin E, Szaflarski J. Importance of Monitoring Clobazam and N-Desmethylclobazam Levels in Treatment with Cannabidiol (CBD) for Epilepsy (S14.001). Neurology. 2016 Apr. 5; 86(16 Supplement):S14.001.
45. In-Common Laboratories. Resources—OLA—In-Common Laboratories [Internet]. In-Common Laboratories. 2015 [cited 2017 Aug. 22]. Available from: http://www.hicl.on.ca/icl-ola.shtml
46. Panelli R J, Kilpatrick C, Moore S M, Matkovic Z, D'Souza W J, O'Brien T J. The Liverpool Adverse Events Profile: relation to AED use and mood. Epilepsia. 2007 March; 48(3):456-63.
47. Morita D A, Glauser T A, Modi A C. Development and validation of the Pediatric Epilepsy Side Effects Questionnaire. Neurology. 2012 Sep. 18; 79(12):1252-8.
48. General Standards—NINDS Common Data Elements [Internet]. [cited 2017 Jul. 31]. Available from: https://www.commondataelements.ninds.nih.gov/General.aspx#tab=Data Standards
49. Grinnon S T, Miller K, Marler J R, Lu Y, Stout A, Odenkirchen J, et al. National Institute of Neurological Disorders and Stroke Common Data Element Project—approach and methods. Clin Trials Lond Engl. 2012 June; 9(3):322-9.
50. Ferguson L, Scheman J. Patient global impression of change scores within the context of a chronic pain rehabilitation program. J Pain. 2009 Apr. 1; 10(4):S73.
51. Sajobi T T, Jette N, Zhang Y, Patten S B, Fiest K M, Engbers J D T, et al. Determinants of disease severity in adults with epilepsy: Results from the Neurological Diseases and Depression Study. Epilepsy Behay. 2015 Oct. 1; 51:170-5.
52. Speechley K N, Sang X, Levin S, Zou G Y, Eliasziw M, Smith M L, et al. Assessing severity of epilepsy in children: preliminary evidence of validity and reliability of a single-item scale. Epilepsy Behav E B. 2008 August; 13(2):337-42.
53. Chan C J, Zou G, Wiebe S, Speechley K N. Global assessment of the severity of epilepsy (GASE) Scale in children: Validity, reliability, responsiveness. Epilepsia. 2015 December; 56(12):1950-6.
54. Cramer J A, Perrine K, Devinsky O, Bryant-Comstock L, Meador K, Hermann B. Development and cross-cultural translations of a 31-item quality of life in epilepsy inventory. Epilepsia. 1998 January; 39(1):81-8.
55. Goodwin S W, Lambrinos A I, Ferro M A, Sabaz M, Speechley K N. Development and assessment of a shortened Quality of Life in Childhood Epilepsy Questionnaire (QOLCE-55). Epilepsia. 2015 June; 56(6):864-72.
56. WHO|WHO Quality of Life-BREF (WHOQOL-BREF) [Internet]. WHO. Available from: http://www.who.int/substance_abuse/research_tools/whoqolbref/en/
57. Buysse D J, Reynolds C F, Monk T H, Berman S R, Kupfer D J. The Pittsburgh sleep quality index: A new instrument for psychiatric practice and research. Psychiatry Res. 1989 May 1; 28(2):193-213.
58. Spitzer R L, Kroenke K, Williams J B W, Lowe B. A brief measure for assessing generalized anxiety disorder: the GAD-7. Arch Intern Med. 2006 May 22; 166(10):1092-7.
59. Gilliam F G, Barry J J, Hermann B P, Meador K J, Vahle V, Kanner A M. Rapid detection of major depression in epilepsy: a multicentre study. Lancet Neurol. 2006 May 1; 5(5):399-405.
60. Rush A J, Trivedi M H, Ibrahim H M, Carmody T J, Arnow B, Klein D N, et al. The 16-Item Quick Inventory of Depressive Symptomatology (QIDS), clinician rating (QIDS-C), and self-report (QIDS-SR): a psychometric evaluation in patients with chronic major depression. Biol Psychiatry. 2003 Sep. 1; 54(5):573-83.
61. Derogatis L R, Melisaratos N. The Brief Symptom Inventory: an introductory report. Psychol Med. 1983 August; 13(3):595-605.
62. Leon A C, Olfson M, Portera L, Farber L, Sheehan D V. Assessing psychiatric impairment in primary care with the Sheehan Disability Scale. Int J Psychiatry Med. 1997; 27(2):93-105.
63. R. Gallily, Z. Yekhtin, and L. Ond, “Overcoming the Bell—Shaped Dose—Response of Cannabidiol by Using Cannabis Extract Enriched in Cannabidiol,” Pharmacol. Pharm., vol. 6, pp. 75-86, 2015.
64. Kwan, P., Arzimanoglou, A., Berg, A. T., Brodie, M. J., Allen Hauser, W., Mathern, G., . . . French, J. (2009). Definition of drug resistant epilepsy: Consensus proposal by the ad hoc Task Force of the ILAE Commission on Therapeutic Strategies. Epilepsia, 51(6), 1069-1077.

1. A method for treating seizures in a subject having epilepsy, the method comprising administering a pharmaceutical composition comprising cannabidiol (CBD) and Δ⁹-tetrahydrocannabinol (THC) to the subject, wherein the ratio of CBD to THC in the pharmaceutical composition is between about 14:1 and about 17:1.

2. The method of claim 1, wherein the ratio of CBD to THC in the pharmaceutical composition is between about 15:1 and about 16:1.

3. The method of claim 1, wherein the pharmaceutical composition comprises a Cannabis extract.

4. The method of claim 3, wherein the Cannabis extract is from the Avidekel™ cultivar.

5. The method of claim 3, wherein the Cannabis extract is a whole plant extract.

6. The method of claim 1, wherein the pharmaceutical composition further comprises cannabiochromene (CBC).

7. The method of claim 1, wherein the pharmaceutical composition further comprises at least one terpene.

8. The method of claim 1, wherein the subject is an adult.

9. The method of claim 1, wherein the seizures are drug-resistant seizures.

10. The method of claim 1, wherein the subject has at least one comorbidity of epilepsy and the pharmaceutical composition treats the at least one comorbidity.

11. The method of claim 10, wherein the at least one comorbidity comprises at least one of depression, anxiety, and Autism Spectrum Disorder.

12. The method of claim 1, wherein the pharmaceutical composition is administered concomitantly with at least one other anti-seizure medication.

13. The method of claim 1, wherein the subject is not concurrently taking another anti-seizure medication and the pharmaceutical composition is administered to the subject as a monotherapy.

14. The method of claim 1, wherein the pharmaceutical composition is administered to the subject at a CBD dosage of between about 100 mg/day and about 300 mg/day.

15. The method of claim 1, wherein the pharmaceutical composition is administered to the subject at a THC dosage of between about 5 mg/day and about 20 mg/day.

16. The method of claim 1, wherein the pharmaceutical composition is administered orally.

17. The method of claim 1, wherein the pharmaceutical composition is administered parenterally.

18. A kit comprising a pharmaceutical composition comprising CBD and THC in a container and prescribing information that includes instructions for taking the pharmaceutical composition to treat seizures, wherein the ratio of CBD to THC in the pharmaceutical composition is between about 14:1 and about 17:1.

19. The kit of claim 18, wherein the prescribing information includes instructions for use of the pharmaceutical composition with at least one other medication.

20. The kit of claim 19, wherein the at least one other medication comprises an anti-seizure medication.
